(19) United States
(12) Patent Application Publication
(10) Pub. No.: US 2002/0110910 A1
(43) Pub. Date: Aug. 15, 2002

(54) Isolation procedure and optimized media solution to enhance long-term survival of cells

(75) Inventors: Judith K. Gwathmey, Cambridge, MA (US); Federica del Monte, Cambridge, MA (US); Roger J. Hajjar, Cambridge, MA (US); Georges E. Haddad, Rockville, MD (US)

Correspondence Address: LAHIVE & COCKFIELD, 28 STATE STREET, BOSTON, MA, 02109, US

(73) Assignee: Gwathmey, Inc., Cambridge, MA

(21) Appl. No.: 09/990,705

(22) Filed: Nov. 21, 2001

Related U.S. Application Data
(60) Provisional application No. 60/252,657, filed on Nov. 22, 2000.

(51) Int. Cl. C12N 5/02

(52) U.S. Cl. 435/325; 435/40.5

(57) ABSTRACT

Isolation, enhanced yield, and maintenance (e.g., culture) methods for cells, e.g., cardiomyocytes, which maintain the structural and functional characteristics of freshly isolated cells are disclosed. Such methods can be used for developing long-term maintenance/cultures of cardiac myocytes which can be used in, for example, in vitro gene transfer, protein expression studies, and small molecule or drug screening, testing, toxicological study. Optimized media solutions to enhance long-term survival of acutely isolated cells are also provided.

### RELATED APPLICATIONS

This application claims priority to U.S. Provisional Application No. 60/252,657 filed on Nov. 22, 2000.

### BACKGROUND OF THE INVENTION

Heart failure is a debilitating clinical syndrome that occurs when the heart is unable to pump an adequate supply of blood to meet the metabolic needs of the different organs of the body. (Senni, M., et al., Archives of Internal medicine (1999) 159(1):29-34). Congestive heart failure (CHF) may result from various etiologies such as coronary artery disease, hypertension, diabetes, viral infections, substance abuse or heart valve defects, congenital heart diseases, which ultimately affects heart muscle as well as from unknown etiologies (idiopathic cardiomyopathy). Visits to a physician's office for CHF increased from 1.7 million in 1980 to 2.9 million in 1993. Heart failure is among the most serious health problems facing the U.S. health care system today. (Senni, et al.). CHF affects more than five million Americans today, with approximately 400,000 new cases reported each year. CHF is a progressive disease and half of the patients with CHF die within five years of diagnosis. CHF is now implicated in approximately 260,000 deaths each year in the U.S. Between 1979 and 1994, hospital discharges for CHF rose from 377,000 to 874,000. (Haldeman, G. A., et al., Am. Heart J. (1999) 137(2):352-360). More than 65,000 persons with CHF receive home care each year. (Haldeman, G. A., et al.). The burden of CHF is expected to get much worse because of increasing life spans and a growing aging population and paradoxically as a result of a better treatment for myocardial infarction, which reduces early mortality for acute events with the result of a concomitant enhancement of the chance of developing heart failure.

Over the past decade considerable progress has been made in the treatment of Class I-III heart failure patients using angiotensin converting enzyme inhibitors (ACE-inhibitors) and β-adrenergic receptor antagonists (β-blockers). (Cleland, J. G. and A. Clark, Am. J. Cardiol. (1999) 83(5B):1 12D-119D). β-blockers appear to offer additional benefit to subjects treated with ACE-inhibitors, but β-blockers are not suited for subjects with decompensated or recently decompensated heart failure. Treatment of these individuals, which constitute 10- 15% of the heart failure population at any one time and a much higher cumulative population over the life span of heart failure patients, is limited to admitting them to the hospital or delivering an intravenous inotropic agent. Either approach is costly and difficult for the individual patient. For these reasons, more effective treatments are needed for advanced heart failure patients, particularly those who are too far advanced for β-blocker therapy.

It is well established that in heart failure and associated cardiac hypertrophy, cardiomyocytes respond to the numerous pathologic stimuli by increasing in cell size and activating expression of fetal cardiac genes, the so-called “fetal gene program,” not typically expressed in adult cardiomyocytes. (Force, T. et al., Gene Expr. (1999) 7(4-6):337-48). Although the effects of cardiac hypertrophy and heart failure have been extensively documented, the underlying molecular mechanisms that link the hypertrophic stimuli delivered to the cardiomyocyte cell to the cell's response in the form of changes in gene expression remains to be elucidated. This is especially true now that transcript profiling using microarray display can identify genes that are differentially expressed in failing versus nonfailing hearts. (Lee, P. S. and K. H. Lee, Curr. Opin. Biotechnol. (1000) 11(2):171-5; Dutt, M. J. and K. H. Lee, Curr. Opin. Biotechnol. (2000) 1 (2):176-9; van Hal, N. L., et al., J. Biotechnol. (2000) 78(3):271-80; Ryu, D. D. and D. H. Nam, Biotechnol. Prog. (2000) 16(1):2-16; Claverie, J. M., Hum. Mol. Genet. (1999) 8(10):1821-32). To elucidate the mechanisms of action of these genes, knockout or transgenic mouse models can be used. However, the relevance to human disease is always in question with these types of models. Experimental models in rodents have significant differences in terms of their subcellular apparatus when compared to human cardiac cells. These include differences in calcium regulatory and myofibrillar proteins, (Hardings, S. E., et al., Cardioscience (1990) 1(1):49-53; del Monte, F., et al., Cardioscience (1993) 4(3):185-91). Therefore, targeting genes in experimental models may have significantly different effects than in human cardiac cells. (Harding, S. E., et al.; del Monte, F., et al.).

Animals are used in pre-clinical testing of small molecules and drugs. Before submission to regulatory agencies, such as the FDA, Good Laboratory Practices must be used to perform animal experimentation. The relatively new field of cardiac genomics has attempted to identify the factors responsible for the development and progression of cardiac dysfunction in patients and animals with heart failure. Using the constantly evolving microchip technology, many large and medium sized pharmaceutical companies have been working to identify genetic changes in banked tissue from animals and patients at various stages of heart failure. A limitation of this work to date has been its emphasis on a “single point in time” analysis of tissue from a given individual or animal with little attention being paid to longitudinal and progressive changes in an individual animal or patient or to important inter-species differences that may help prioritize in importance the myriad number of genetic modifications that occur in this disease state. Moreover, the majority of the studies that have been performed to date have involved the use of tissue obtained from animals or patients with ischemic etiologies of heart failure; an increasing amount of evidence is accumulating to suggest that non-ischemic cause of heart failure may demonstrate a different pathophysiology which could reasonably be expected to provide a unique pattern of genomic alterations.

In order to better understand the effects of small molecules, whether targeted for the heart or not, the specific signaling and control pathways influencing gene expression at the transcriptional and post-transcriptional levels, therapeutic drug targeting based on gene profiling should be performed in human and animal cardiomyocytes. Similarly, in vitro testing for cardiac targeted small molecules and gene therapies, specificity, efficacy, and toxicity can best be measured, analyzed, and validated in isolated single myocytes. Accordingly, a need exists for reliable and cost-effective long-term maintenance of human, as well as animal, cardiomyocytes.

Challenges involved in cell culture for adult differentiated cardiac myocytes have been the short durations for culture before de-differentiation and change in morphological and function phenotypes occur. Several laboratories have refined the cultured adult rat ventricular myocyte model system (Claycomb, W. C. and M. C. Palazzo, Dev. Biol. (1980) 80(2):466-82; Eckel, J. G. et al., Am. J. Physiol. (1985) 249(2 Pt 2):H212-21) over the last 15 years. The few investigators isolating human myocytes report poor yields and viability as a result of the current isolation procedures and have been challenged in the review process about potential selection bias in cells that survive the procedure. This state of research necessitates the study of hundreds of cells in order to get a better handle on whether the cells reflect global cardiac dysfunction and disease. Furthermore, if a sufficient number of cells from the same hearts were available, it would be possible to conduct studies in multiple laboratories with the sharing of basic data and the simultaneous screening of small molecules, drugs, and gene targets. Also, by enabling access to multiple investigators, biotechnology and pharmaceutical industries, not only can intraobserver variability be addressed, but also interobserver variability and reproducibility can be addressed. However, most investigators only study one to two myocytes per heart. Currently, because of limited access to human tissues, investigators in both the academic and commercial industries have not in a systematic manner worked to improve their isolation or culturing methods of human myocytes.

The most recent methods for culturing adult rat cardiomyocytes derive from the well-described methodologies of Ellingsen et al. (Ellingsen, O. et al., Am. J. Physiol. (1993) 265(2 Pt 2):H747-54). Cultured myocytes are grown in serum free medium with minimal growth factors so that the myocytes appear morphologically to be almost identical to freshly dissociated adult rat myocytes. There are subtle changes in the myocyte contractile properties over the first 3 days in culture. Many relevant aspects for the system are stable for at least six days. For example, Ellingsen et al. demonstrated that abundance of the alpha and beta MHC isogene mRNA remains constant over eight days in culture in this system. Skeletal alpha actin mRNA abundance decreases by day eight whereas cardiac alpha actin mRNA levels remain stable. Calcium channel function (peak I_{Ca}, activation and inactivation kinetics) is stable for at least six days.

The cultured adult myocyte system offers substantial advantages over cultured neonatal rat ventricular myocytes for study of proteins critical to excitation-contraction (E-C) coupling and testing of small molecules (e.g. drugs). Adult myocytes have substantially different E-C properties than neonatal cells. More recently, a technique to culture adult murine isolated cardiac myocytes while maintaining their morphological integrity for greater than 3 days was developed by Zhou et al. (Zhou, Y. -Y., et al., Am. J. Physiol. (2000) 279:429-436). These investigators have modified current enzymatic methods to improve adult mouse cardiomyocytes yield and quality and have developed a practical method for short-term culture of these isolated myocytes that retains their morphological as well as physiological integrity. Moreover, they demonstrated the feasibility of adenovirus-mediated gene transfer in such cultured myocytes from both wild-type and genetically engineered mouse hearts. These new technical developments provide a set of powerful tools for acute gene engineering in single cells, for phenotyping transgenic or knockout models at the cellular and subcellular levels, and for combining the approaches of whole animal and single-cell gene manipulations.

However, despite these advances in experimental models of cardiac cell culturing, an adequate method for enhancing human cardiomyocyte yield and cell maintenance is still lacking, as well as long term maintenance of animal myocytes.

### SUMMARY OF THE INVENTION

The present invention establishes discovery, testing, analysis, and validation platforms for screening gene targets, as well as small molecules, drugs, and compounds for proof of concept, testing, screening, and toxicological study in myocytes maintained without dedifferentiation for extended periods. The present invention optimizes cell isolation procedures enhancing yield of viable myocytes and maintenance (e.g., culturing media) media conditions for human and animal myocytes in order to attain long term survival of myocytes without de-differentiation and expression of fetal gene programs. Accordingly, with the present methods and culture media, the functional integrity of the cardiac myocyte is maintained along with the preservation of adult expression profiles of key regulatory proteins involved in excitation-contraction coupling. Other advantages of the current invention include substantial reduction in the need for human heart harvesting, animal sacrifices, and reductions in the costs associated with preparing fresh myocytes. Another advantage of the current invention is that large numbers of genes, small molecules, and drugs can be screened in cells from the same heart. Another advantage of the current invention is the establishment of a screening platform for discovery, testing, analysis and validation of safety, efficacy, and toxicology of agents whether targeted for the myocyte or not.

The invention features methods for isolating and maintaining, e.g., culturing cells, e.g., myocytes. The present invention also features methods for enhancing the yield of viable myocytes, long term survival and maintenance, e.g., culture of isolated cells.

In one embodiment, the invention features methods of isolating cells, e.g., cardiomyocytes, including the steps of obtaining a tissue sample from a subject, e.g., a vertebrate or non-vertebrate subject, and successively exposing the tissue to a first solution with decreasing amounts of CaCl₂. The first solution further includes NaCl, HEPES, MgCl₂, KCl, and sugar at a pH of approximately 7.4. The present methods also include the steps of disassociating the tissue with an enzyme solution and repeatedly resuspending the disassociated tissue in a second solution with increasing amounts of CaCl₂. The second solution further including Earle's modified salt, L-glutamine, sodium bicarbonate, sodium pentothenate, creatine, taurine, ascorbic acid, HEPES, fetal bovine serum, an antibiotic, and a fatty acid, at a pH of approximately 7.4.

In another embodiment, the invention features methods of isolating cells, e.g., cardiomyocytes, including the steps of obtaining a tissue sample from a subject, e.g., a vertebrate or invertebrate subject, and successively exposing the tissue to a first solution with decreasing amounts of CaCl₂ at approximately 37° C. The first solution further includes approximately 140 mM NaCl, approximately 10 mM HEPES, approximately 1 mM MgCl₂, approximately 5.4 mM KCl, and approximately 10 mM sugar at a pH of approximately 7.4. With the addition of an enzyme to the first solution, the present methods also include the step of disassociating the tissue in this solution to form disassociated cells and repeatedly resuspending the disassociated cells in a second solution with increasing amounts of CaCl₂. The second solution further includes Earle's modified salt, L-glutamine, sodium bicarbonate at approximately 1250 mg/l, sodium pentothenate, creatine at approximately 328 mg/500ml, taurine at approximately 312 mg/500ml, ascorbic acid at approximately 8.8 mg, HEPES at approximately 2.383 g/500ml, fetal bovine serum at approximately 10% v/v, an antibiotic at approximately 5% v/v, and a fatty acid at approximately 1 μM at a pH of approximately 7.4. The methods of the present invention also include the steps of incubating the isolated cells in a mixture of carbon dioxide and air at approximately 37° C. and re-suspending the isolated cells approximately every 24 hours in the second solution.

In yet another embodiment, the second solution can be used to cultivate isolated cells, e.g., cardiomyocytes, including the steps of resuspending the isolated cells approximately every 24 hours in the second solution. In still another embodiment, the second solution can be used as maintenance or culture media for cells, e.g., cardiomyocytes.

In another embodiment, the invention features methods of isolating cells, e.g., cardiomyocytes, including the steps of obtaining a tissue sample from a subject, e.g., a vertebrate or invertebrate subject, chopping the tissue, and incubating the tissue in a first solution. The first solution includes calcium, salts, magnesium sulfate, pyruvate, glucose, taurine, HEPES, and nitrilotriacetic acid. With the addition of an enzyme, e.g, collagenase, to the first solution, the methods further include the steps of incubating the tissue in this solution and centrifuging the tissue to obtain isolated cells.

In still another embodiment, the invention features methods of isolating cells, e.g., cardiomyocytes, including the steps of obtaining a tissue sample from a subject, e.g., a vertebrate or invertebrate subject, chopping the tissue, and incubating the tissue in a first solution. The first solution includes approximately 1-2 μM CaCl₂, approximately 120 mM NaCl, approximately 5.4 mM KCl, approximately 5 mM MgSO₄, approximately 5 mM pyruvate, approximately 20 mM glucose, approximately 20 mM taurine, approximately 10 mM HEPES, and approximately 5 mM nitrilotriacetic acid, at a pH of approximately 6.96. The methods further include the steps of shaking the tissue at approximately 37° C. for approximately 12 minutes, bubbling approximately 100% O₂ through the solution, incubating the tissue in a second solution comprising approximately 1-2 μM CaCl₂, approximately 30 μM NaCl, approximately 5.4 mM KCl, approximately 5 mM MgSO₄, approximately 5 mM pyruvate, approximately 20 mM glucose, approximately 20 mM taurine, approximately 10 mM HEPES, and 4 U/ml of a digestive enzyme, incubating the solution in a third solution comprising approximately 1-2 μM, approximately 30 μM NaCl, approximately 5.4 mM KCl, approximately 5 mM MgSO₄, approximately 5 mM pyruvate, approximately 20 mM glucose, approximately 20 mM taurine, approximately 10 mM HEPES, and 4 U/ml of a digestive enzyme, and centrifuging the tissue to obtain isolated cells.

In a preferred embodiment, embodiment, the invention features methods of isolating cells, e.g., cardiomyocytes, including the steps of obtaining a tissue sample from a subject, e.g., a vertebrate or invertebrate subject, chopping the tissue, and incubating the tissue in a first solution. The first solution includes approximately 1-2 μM Ca²⁺, 120 mM NaCl, 5.4 mM KCl, B 5 mM MgSO₄, 5 mM pyruvate, 20 mM glucose, 20 mM taurine, 10 mM HEPES, and 5 mM nitrilotriacetic acid, at a pH of 6.96. The methods further include the steps of shaking the tissue at 37° C. for 12 minutes, bubbling 100% O₂ through the solution, incubating the tissue in a second solution comprising Ca²⁺, 50 μM Ca²⁺, 120 mM NaCl, 5.4 mM KCl 5.4, 5 mM MgSO₄, 5 mM pyruvate, 20 mM glucose, 20 mM taurine, approximately 10 mM HEPES, and 4 U/ml of a digestive enzyme, incubating the solution in a third solution comprising 50 μM Ca²⁺, 120 mM NaCl, 5.4 mM KCl 5.4, 5 mM MgSO₄, 5 mM pyruvate, 20 mM glucose, 20 mM taurine, approximately 10 mM HEPES, and 400 U/ml of a digestive enzyme, and centrifuging the tissue to obtain isolated cells.

### BRIEF DESCRIPTION OF THE FIGURES

FIG. 1 shows recordings from cardiomyocytes isolated from donor nonfailing heart and from failing heart infected with either Ad.GFP or adenoviral vector targeting SERCA2a (Ad.SERCA2a), stimulated at 1 Hz at 37° C. The failing cell had a characteristic decrease in contraction and prolonged relaxation along with a prolonged Ca²⁺ transient. Overexpression of SERCA2a in the failing cardiomyocyte normalized these parameters.

FIG. 2 is a table, which shows the contraction velocity, relaxation and systolic and diastolic Ca²⁺ concentrations in human cardiomyocytes from a donor nonfailing heart and from failing heart infected with either Ad.GFP or Ad.SERCA2a, stimulated at 1 Hz at 37° C.

FIG. 3 shows recordings from the same cardiomyocytes as in FIG. 2 stimulated at increasing frequencies. The failing cardiomyocyte demonstrates a decrease in contraction amplitude and an increase in diastolic tone and Ca²⁺. Overexpression of SERCA2a restored the frequency-dependent increase in contraction amplitude and mitigated an increase in diastolic Ca²⁺ and decrease in cell length.

FIG. 4 (cell #1) shows intracellular calcium concentration of a freshly isolated (day 1) adult rat ventricular myocyte. Normal calcium transient and calcium distribution are present.

FIG. 5 (cell #2) shows intracellular calcium concentration of an adult rat ventricular myocyte after 30 hours in culture (day 2). The cell maintained normal intracellular calcium concentrations and distribution.

FIG. 6 (cell #3) is the same as cell #2. The same cell exposed to maintenance conditions has normal calcium transient and calcium distribution is present.

FIG. 7 (cell #4) is the same as cell #2. The same cell exposed to maintenance conditions has normal calcium transient and calcium distribution is present.

FIG. 8 (cell #5) shows the intracellular calcium concentration of an adult rat ventricular myocyte after three days in maintenance media. The cell has normal calcium transients and calcium distribution as seen in freshly dissociated cells.

FIG. 9 (cell #6) is the same as cell #5. The same cell exposed to maintenance conditions has normal calcium transient and calcium distribution is present.

FIG. 10 (cell #7) is the same as cell #5. The same cell exposed to maintenance conditions has normal calcium transient and calcium distribution is present.

FIG. 11 (cell #8) shows the intracellular calcium concentration of an another adult rat ventricular myocyte after 30 hours in maintenance media). The cell has normal calcium distribution and intracellular calcium levels.

FIG. 12 (cell #9) shows the intracellular calcium concentration of another adult rat ventricular myocyte after 54 hours in maintenance media. Normal intracellular calcium concentration and distribution is maintained.

FIG. 13 (cell #10) is the same as cell #9. Normal intracellular calcium concentration and distribution is maintained. Repeated recordings for the same cell shows that the calcium distribution and concentrations are accurate. Furthermore, cellular homeostasis with regard to calcium is maintained and the cells have not been damaged by the addition of the calcium indicator. Calcium is the key intracellular ion in excitation-contraction coupling and is a pivotal marker of the intact state, membrane stability, and viability of the cell.

### DETAILED DESCRIPTION OF THE INVENTION

The present invention provides methods for isolating and maintaining (e.g., culturing) cells, e.g., cardiomyocytes, which enhance yield, the long-term survival rate of the cells and minimize the alterations in the subcellular and structural components of the cells. The present invention also provides optimized maintenance (e.g., culture media) for use in maintaining freshly isolated cells, e.g., cardiomyocytes. As used herein, the terms “cardiomyocytes,” “cardiac myocytes,” “myocytes,” and “cardiocytes” are used interchangeably and refer to the cells found in heart tissue, e.g., ventricular heart tissue. Such cells can be isolated from invertebrates as well as vertebrate animals including rats, mammals, non-mammals, fish, crustacea, avian species and humans and non-human primates.

The methods of the current invention include, for example, a series of mechanical steps, which utilize solutions for disassociating the sample of tissue, e.g., ventricular tissue, into isolated cells and for resuspending the tissue and isolated cells. In particular, the solutions used in the current isolation methods contain varying amounts of calcium chloride (CaCl₂), which have been found useful in enhancing the survival rate of acutely isolated cardiomyocytes.

The method of the present invention is useful in isolating cells, e.g., calcium tolerant human cardiac ventricular myocytes which maintain the structural and functional characteristics of freshly isolated cardiomyocytes over a long period of time, e.g., approximately 72 hours or longer. The methods involve mixing chunks of tissue, e.g., heart muscle, in a solution containing, approximately 1 μM calcium, various salts, magnesium sulfate, pyruvate, glucose, taurine, HEPES, and nitrilotriacetic acid followed by the addition of a digestive enzyme, e.g., a type XXIV protease, such as, Matrix metalloproteinase 2 or 4, and a collagenase, for example, matrix metalloproteinase 1, 3, or 9.

After isolation, the cells are maintained in a culture media comprising, medium DMEM, BSA, ascorbic acid, taurine, carnitine, creatinine, insulin, penicillin G sodium, and an antibiotic. The culture media of the present invention can further comprise M199 medium without calcium chloride anhydrous and D-calcium pantothenate. Sodium pantothenate can be used in order to increase the calcium concentration in a cumulative manner to reduce calcium intolerance. Essential fatty acids or non-essential fatty acids and magnesium (Mg ⁺) can also be added to protect against calcium overload and calcium paradox. Examples of fatty acids for use in the present invention include, among others, omega 3 fatty acids, such as, docosaheanoic acid, eicosapentaenoic acid, eicosatetraynoic acid, or polyunsaturated fatty acids. Cells, e.g., myocardiocytes, isolated by the methods of the current invention can be infected with recombinant adenovirus for in vitro cell culture studies and used for screening small molecules and drugs, genomic profiling, and toxicological study.

The present invention is also drawn to methods for culturing or maintaining cells, e.g., cardiomyocytes, which also utilize solutions developed to enhance the yield and long-term survival rate of these cells. Further, the present invention provides optimized media solutions for maintaining acutely isolated cardiomyocytes.

There are many advantages for using the methods and media solutions of the present invention. For example, the present invention enhances the yield of viable myocytes and extends the survival rate of isolated cells while minimizing the alterations in the subcellular and structural components of the isolated cardiomyocytes, thereby making isolated cardiomyocytes available as the relevant model for the following types of long-term studies: (1) correlation of protein levels with alterations in target genes/message; (2) elucidation of the role of target proteins in disease phenotypes using molecular, biochemical, physiological and histopathological characterization of tissue; (3) identification of endogenous ligands, substrates and regulatory factors relating to drug targets; (4) identification of transcription and translation control points; (5) protein identification, differential expression, and characterization; and (6) small molecule or drug screening for safety, efficacy, toxicity and toxicological profiling (7) research, discovery, testing, validation, and analysis platforms. Other advantages of the current invention include substantial reduction in the need for human heart harvesting, animal sacrifices, and reductions in the number and costs associated with preparing fresh myocytes.

This invention is further illustrated by the following examples, which should not be construed as limiting. The contents of all references, patents and published patent applications cited throughout this application are hereby incorporated by reference.

### EXAMPLES

### Materials and Methods

Hearts: Failing explanted hearts are provided through Massachusetts General Hospital Heart Transplantation program. Donor hearts are obtained through a national procurement agency and flown to the laboratories at Gwathmey Inc. All procedures follow federally regulated guidelines. The molecular grade tissue samples from both diseased and non-diseased hearts are frozen in liquid nitrogen or undergoes fresh dissociation of myocytes. The frozen tissues are then prepared and preserved for RNA/DNA/protein identification and characterization studies. All information obtained on patients and donors are stored on a computerized database. The fresh tissues are used to isolate single ventricular cardiomyocytes (as described below) that can be studied physiologically (as described below). Cell shortening and calcium transients can be measured in these cells (del Monte, F., et al., Cardioscience (1993) 4(3):185-91; del Monte, F., et al., (1999) Circulation 100(23):2308-1 1). Through adenoviral gene transfer, specific pathways can be targeted (Harding, S. E., et al., Cardioscience (1990) 1(1):49-53; del Monte, F., et al., Circulation (1999) 100(23):2308-11). By examining the phenotype of these human cardiomyocytes that are transduced with specific genes or small molecules, pharmacological agents, and drugs, the importance of individual pathways can be evaluated in cardiomyocytes. This allows the elucidation of signaling pathways unique to heart failure pathogenesis as well as the screening of compounds, small molecules, drugs, e.g., elucidation of the signaling and control elements influencing cell function and changes function, e.g., the movement of intracellular calcium and cell shortening.

Human ventricular myocardium is obtained from patients with heart failure secondary due to, e.g., ischemic heart disease, dilated cardiomyopathy or end stage valvular disease, as well as other etiologies, at the time of transplantation. Patient permission is obtained for all samples collected at the time of cardiac transplantation. Tissues received as non-failing hearts from donors can be found not suitable for transplantation for several reasons, e.g., lack of identification of a suitable recipient, blood transfusion while in the emergency room, age of donor, need for resuscitation. All donations are with family approval.

Hearts are handled as if being used for cardiac transplantation. Hearts are placed in a cardioplegia solution e.g., Wisconsin Cardioplegia solution, packed on ice and rushed back to the laboratory (approximately 15 minutes). Samples from all hearts are (1) freeze clamped in liquid nitrogen for later mRNA and protein analyses and (2) with the remainder being placed into to cardioplegia at the site of harvesting for isolation of myocytes. Additional samples are freeze clamped in liquid nitrogen after transport to the laboratory and undergo similar analyses.

Solutions used in the isolation and maintenance of adult Sprague-Dawley (200-250 g) rat ventricular myocytes:

Culture Solution=Solution I (also referred to as Solution IBO)

Five hundred ml of M199 with Earles's modified salt with L-glutamine, sodium bicarbonate, sodium pentothenate, without calcium chloride (anhydrous) and without D-calcium pentothenate. (Formula # 00-01 86DJ, lot # 1082932; Sigma).

Supplemented with:

328 mg Creatine

312 mg Taurine

8.8 mg Ascorbic Acid

2.383 g HEPES

10% Fetal Bovine Serum penicillin-streptomycin

1 μM Eicosapentaenoic Acid (PolyUnsaturated Fatty Acid-PUFA)

Solution I=Culture Solution

Solution IA (also referred to as solution IB100)=Solution I+100 μM Calcium Chloride

Solution IB (also referred to solution IB250)=Solution I+250 μM Calcium Chloride

Solution IC (also referred to as solution IB500)=Solution IBO+500 μM Calcium Chloride

Solution ID (also referred to as solution IA)=Solution I+1 mM Calcium Chloride

Tyrode Solution=Solution II

140 mM NaCl

10 mM HEPES

1 mM MgCl₂

5.4 mM KCl

10 mM Glucose

air Bubbled with 100% O₂

Solution IIA=Solution II+1 mM Solution II

Solution IIB=Solution II (nominal calcium Tyrode solution)

Solution IIC=Solution II+14 mg/100 ml Liberase Blendzyme IV (Collagenase activity

280 Units/90 mg from Roche Diagnostic Corporation)

pH 7.40 for all solutions

### EXAMPLE 1

### Preparation of Culture Media Solutions and Isolation/Maintenance of Cardiomyocytes from Rat

Prepare the following solutions then adjust pH to pH with NaOH or HCl and filter with 0.22μm filters: 250 ml of solutions IA and 100 ml of each solution IB (same as IB0); IB100; IB250; and IB500. 50 ml of each solution IIA, IIB, IIC.

Surgical kit: Curved 20″ scissors (to cut thorax); Sharp 10″ scissors (to cut heart tissue); Curved small forceps (to take heart and attach it on needle); Hemostat to hold rat skin; Alligator to hold the heart by the aorta on needle for perfusion with thread (2:0).

Animal: Sprague-Dawley adult rat.

Procedure: Inject 1 ml of sodium heparin (1000 units/ml) i.p. and wait 10-15 minutes. Anesthetize the rat with 30-40 mg pentobarbital sodium injection, i.p. (50 mg/ml). Cut from the sternum a V-shape and remove the skin to expose the heart. Hold the skin with a hemostat. Cut the heart from beneath at the level of the aorta (make sure to take as much aorta as possible with the heart). Quickly hang the heart with the aorta on the 16 G1 1/2 needle of the perfusion system using the alligator clamp and tighten with a double knot thread (2:0). Perfusion is set at 5 ml/min with a monitored temperature of 36° C.-37° C. It is very important to make sure that the coronaries are perfused by placing the needle-tip 2-3 mm away from the aortic valve.

First, perfuse with solution IIA (1 mM CaCl₂-Tyrode) for 3 minutes. Second, perfuse with solution IIB (0 mM CaCl₂-Tyrode) for 5 minutes (heart should stop beating). Third, perfuse 7-8 minutes with solution IIC (enzymatic solution). The heart should become red-orange in color and swollen in size with loosened texture. Fourth, washout the enzymatic solution by perfusing with solution IIB again for 5 minutes. Cut the ventricles out and place in solution IIB and cut into small pieces of tissue in a suspension. Gently, triturate the suspension solution for better cellular dispersement. Filter the suspension through a nylon mesh (400 μm) take the filtrate, which contains the freshly dissociated ventricular cells. Let cells sediment (by gravity only) for 10 minutes and then discard supernatant. Repeat this step. Resuspend the cell pellets in solution IIB (which is the same as IB0) for 10 minutes, then discard supernatant. Suspend cell pellet in solution IB100 for 10 minutes and then discard supernatant. Suspend cell pellet in solution IB250 for 10 minutes and then discard supernatant. Suspend cell pellet in solution IB500 for 10 minutes and then discard supernatant. Suspend cell pellet in solution IA (culture solution) and seed the cells in culture dishes. Put cultured cells inside a waterjacketed incubator (5% CO₂ and 95% air, at 37° C.). Replenish the cell culture solution (IA) for the cultured cells after 6 hours and then after every 12 hours.

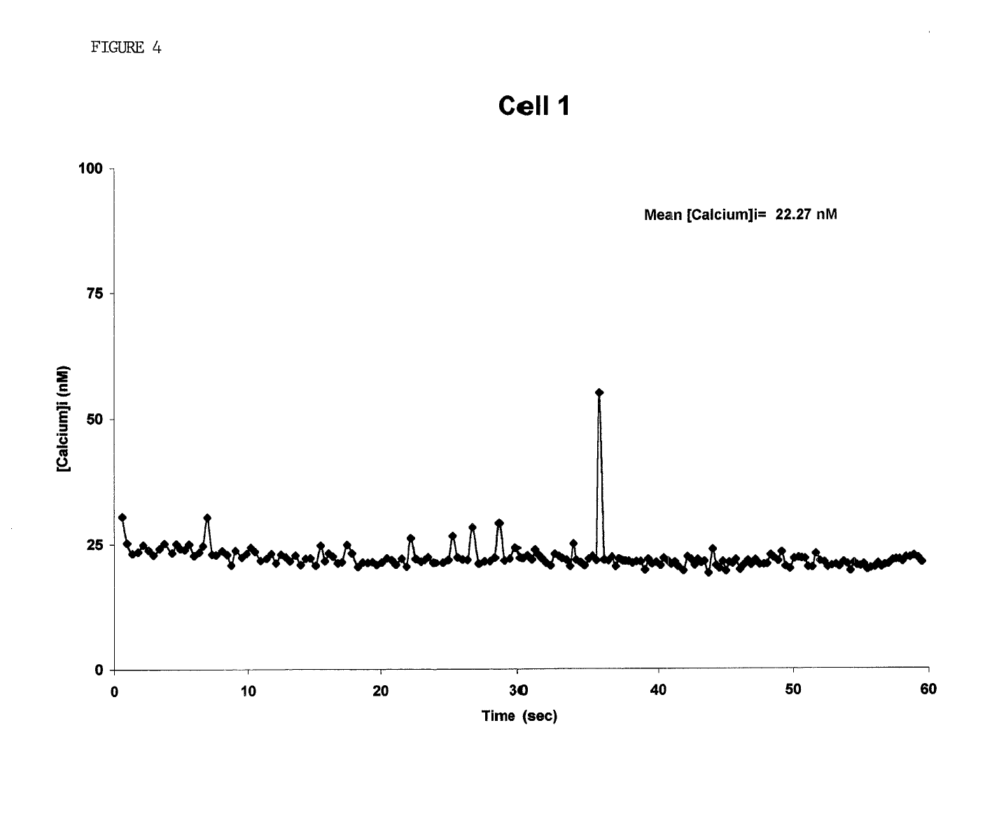

Isolated ventricular myocytes from adult Sprague-Dawley rats according to the above procedure are shown in FIGS. 4-13. These cells were not treated with any antagonist or agonist and some (15%) exhibit spontaneous contraction.

FIG. 4 (cell # 1) shows intracellular calcium concentration of a freshly isolated (day 1) adult rat ventricular myocyte. Calcium imaging data as measured using Fura-II (5 μM). The cell has normal expected calcium distribution and concentrations. Localization in the cytosol and intracellular organelles is as expected in a healthy cell.

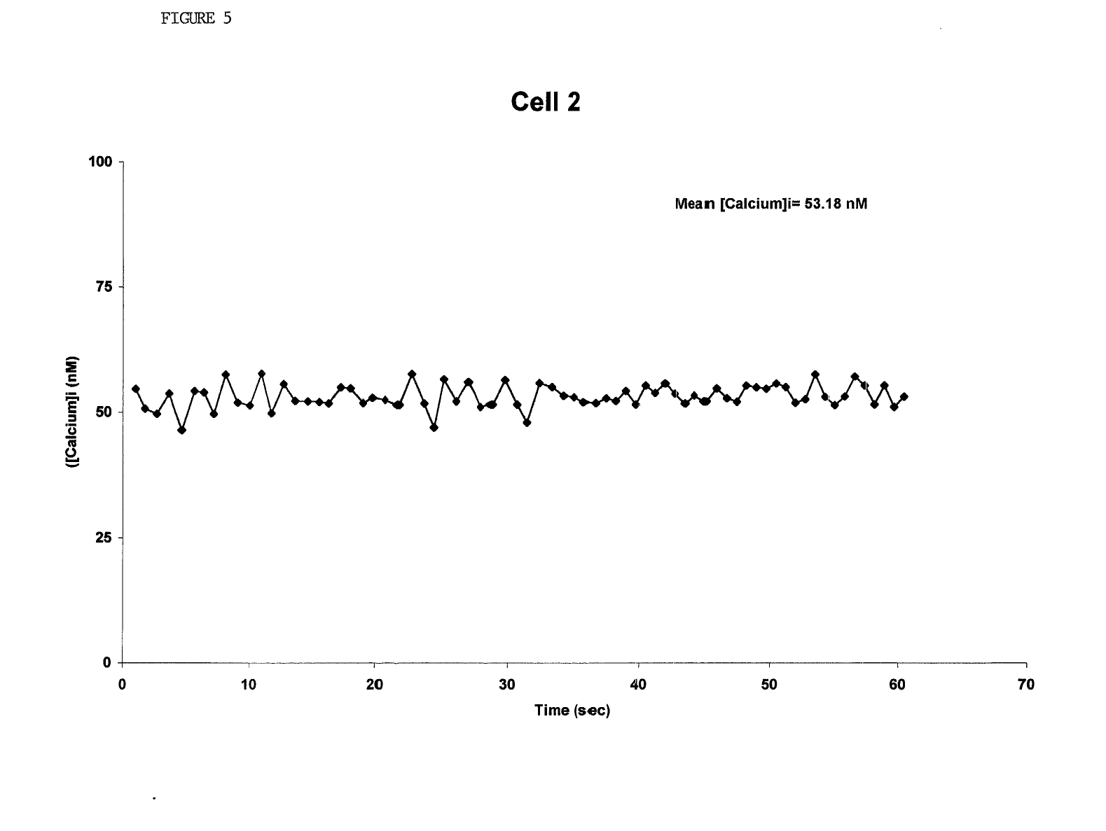

FIG. 5 (cell #2) shows intracellular calcium concentration of an adult rat ventricular myocyte after 30 hours in culture. Calcium imaging data again shows normal distribution intracellularly of calcium as measured using Fura-II (5 μM). This shows that cellular integrity is maintained.

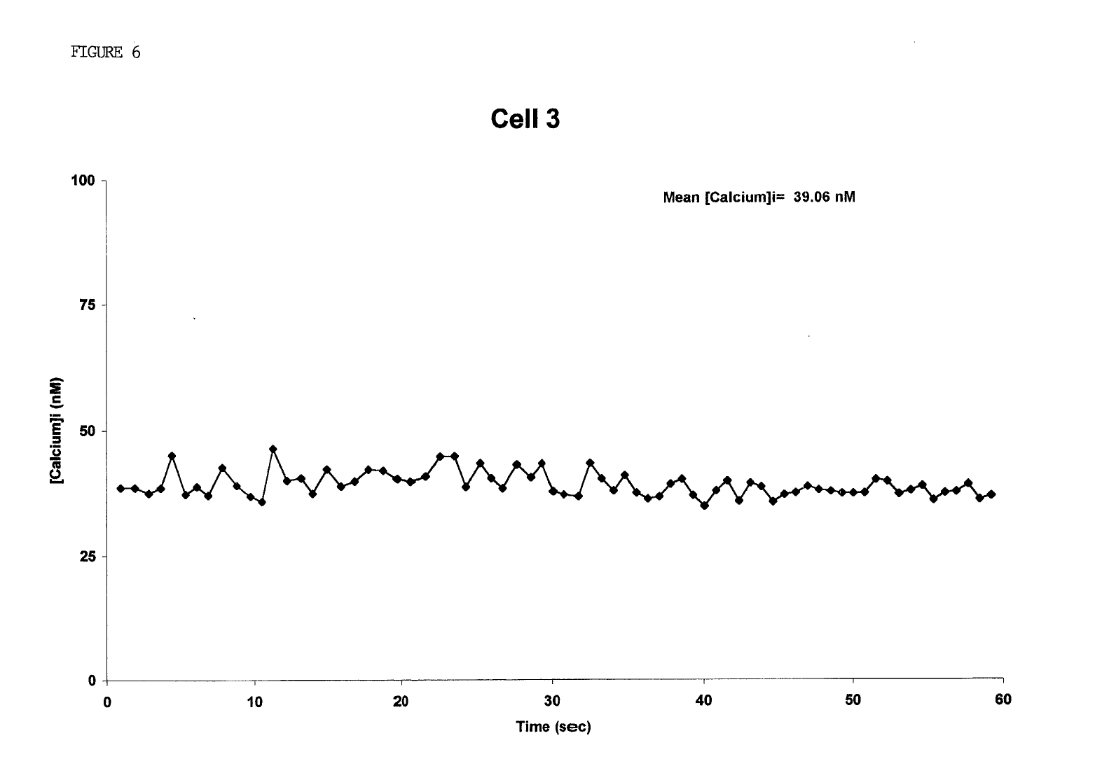

FIG. 6 (cell #3) is the same as cell #2. Calcium imaging data as measured using Fura-II (5 μM) (data not shown) shows. Normal distribution of intracellular calcium is confirmed by repeated imaging of the same cell.

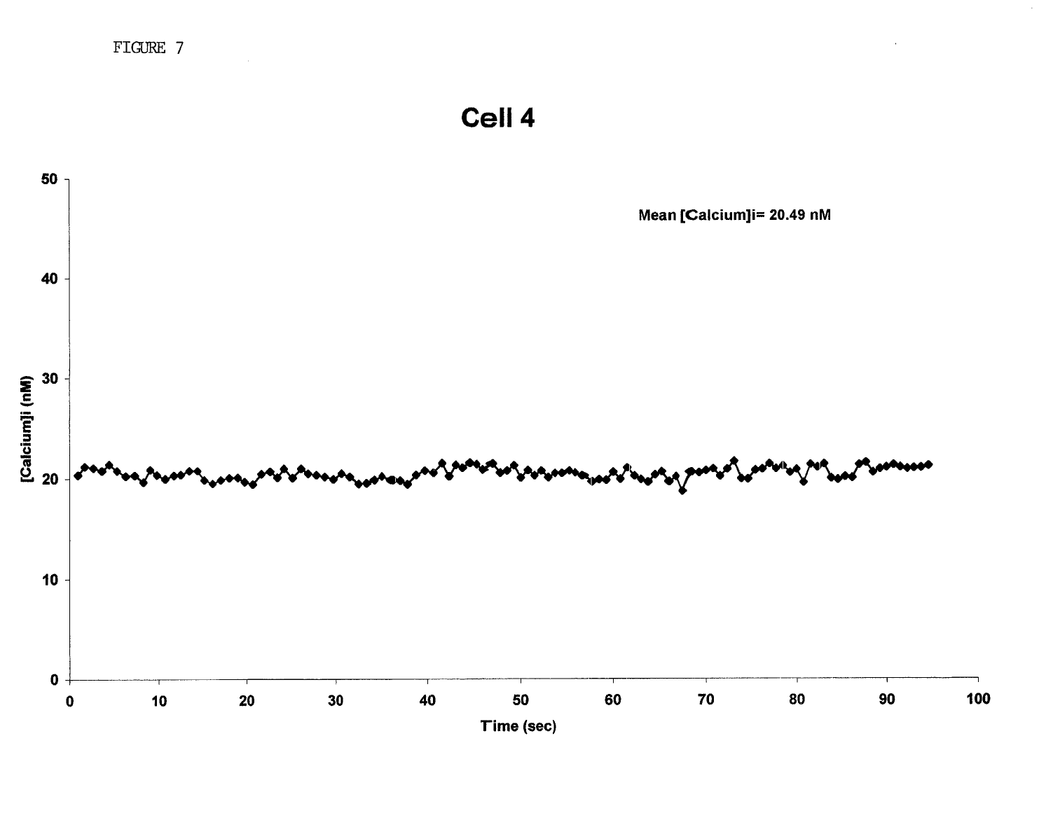

FIG. 7 (cell #4) is the same as cell #2. Calcium imaging data as measured using Fura-II (5 μM) (data not shown) shows normal distribution of intracellular calcium is confirmed by repeated imaging of the same cell.

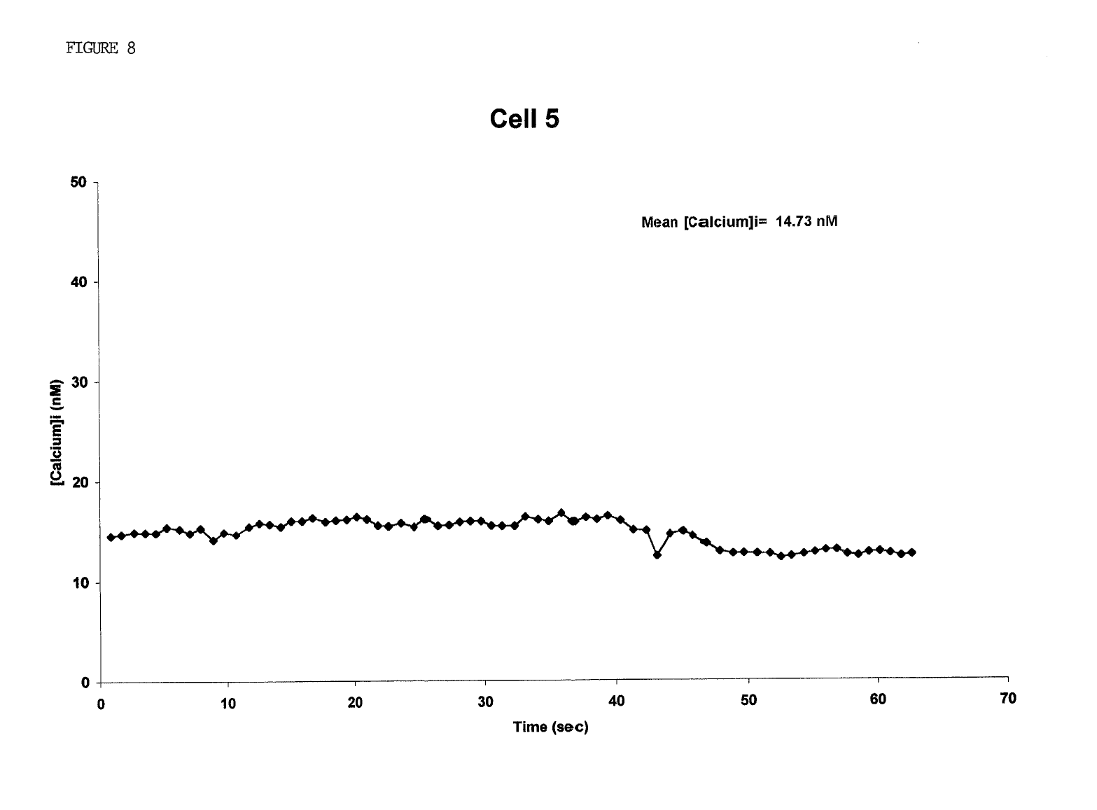

FIG. 8 (cell #5) shows the intracellular calcium concentration of an adult rat ventricular myocyte after 54 hours in culture (day 3). Calcium imaging data as measured using Fura-II (5 μM) (data not shown) shows normal distribution of intracellular calcium is confirmed by repeated imaging of the same cell.

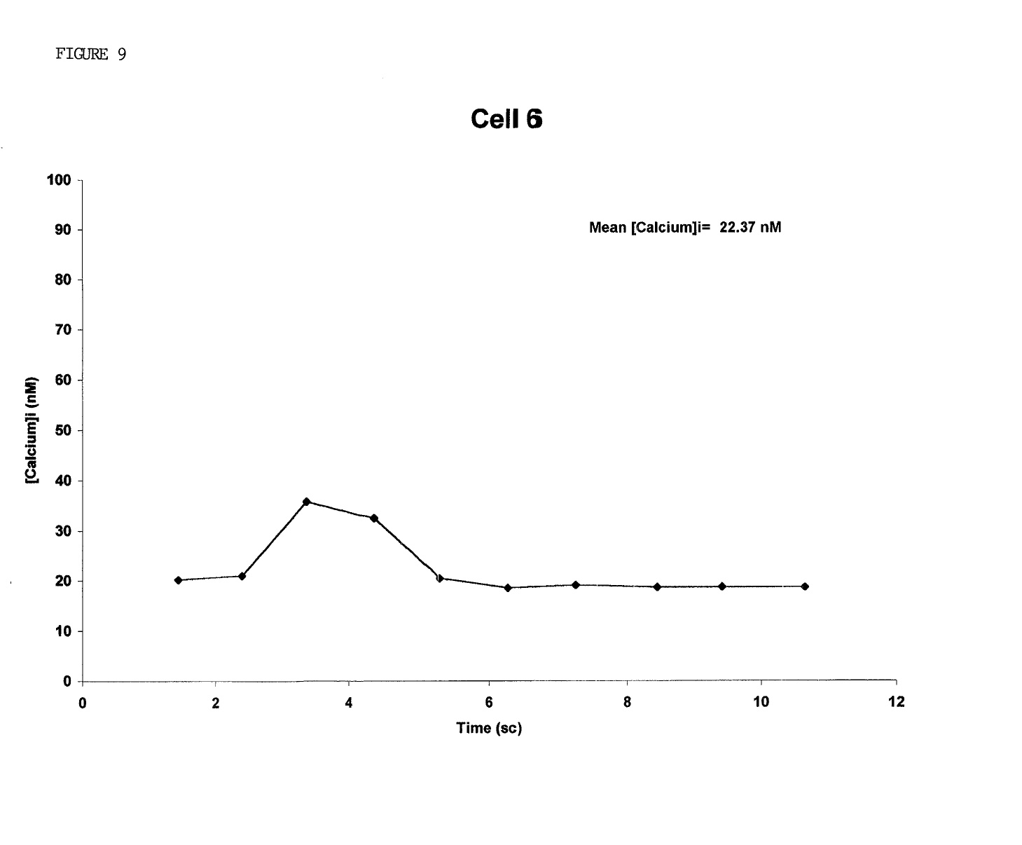

FIG. 9 (cell #6) is the same as cell #5. Calcium imaging data as measured using Fura-II (5 μM) (data not shown) shows that the accuracy of the initial recording is confirmed and that the cell has normal distribution of intracellular calcium.

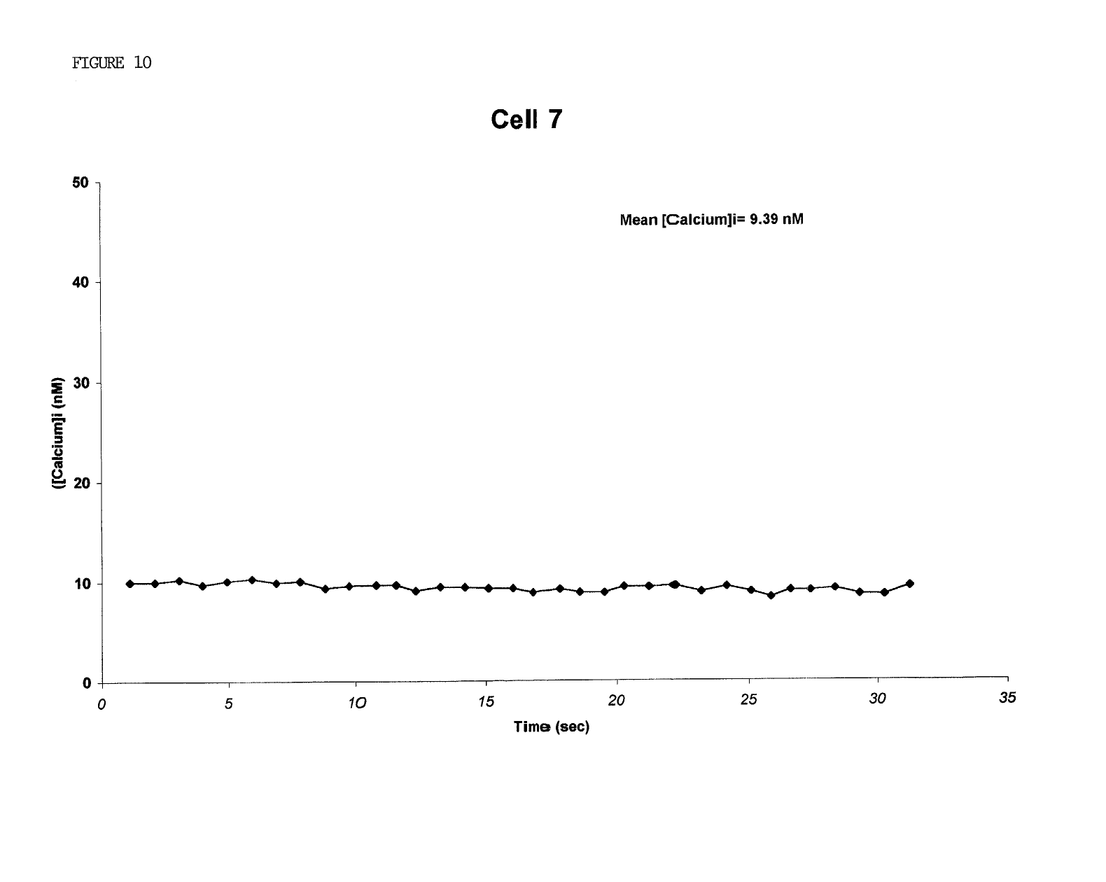

FIG. 10 (cell #7) is the same as cell #5. Calcium imaging data as measured using Fura-II (5 μM) (data not shown) shows normal distribution of intracellular calcium is confirmed by repeated imaging of the same cell. Also, calcium imaging documents that the cells are quiescent with normal structure.

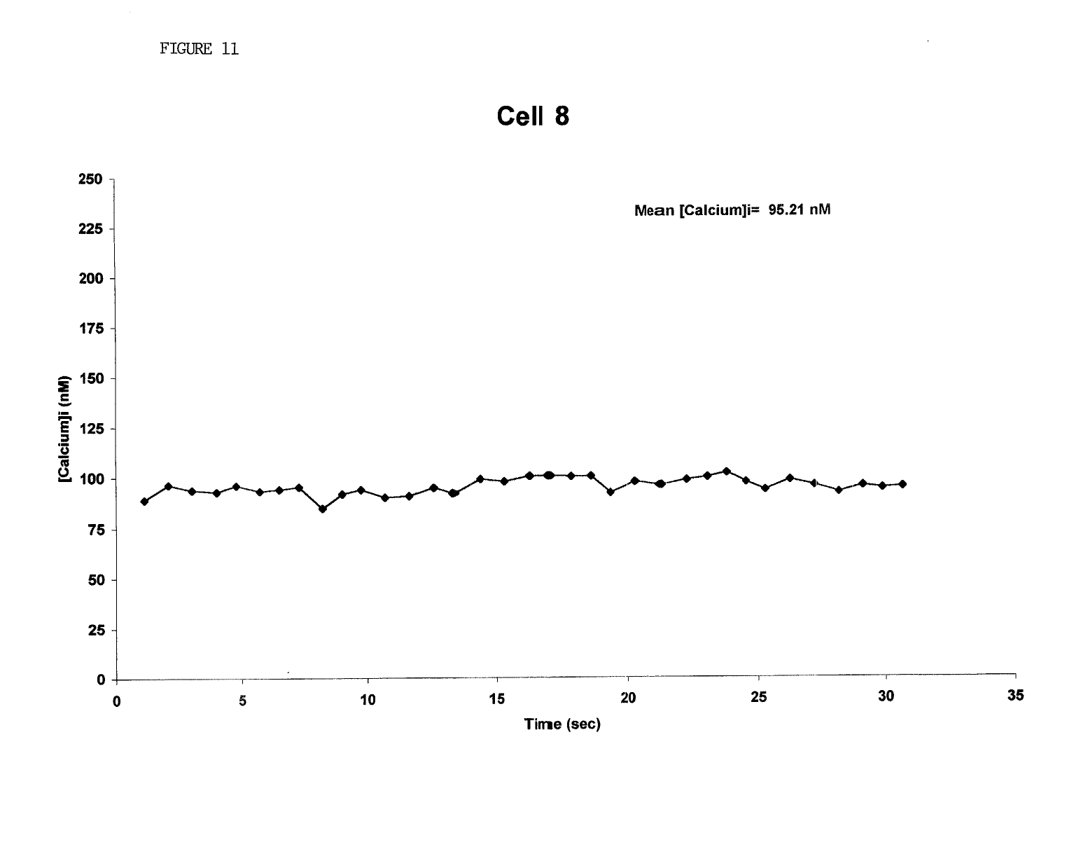

FIG. 11 (cell #8) shows the intracellular calcium concentration of an adult rat ventricular myocyte after 30 hours in media (day 2). Calcium imaging data as measured using Fura-II (5 μM) (data not shown) shows that this cell is spontaneously contracting as evidenced by a shift in the calcium distribution.

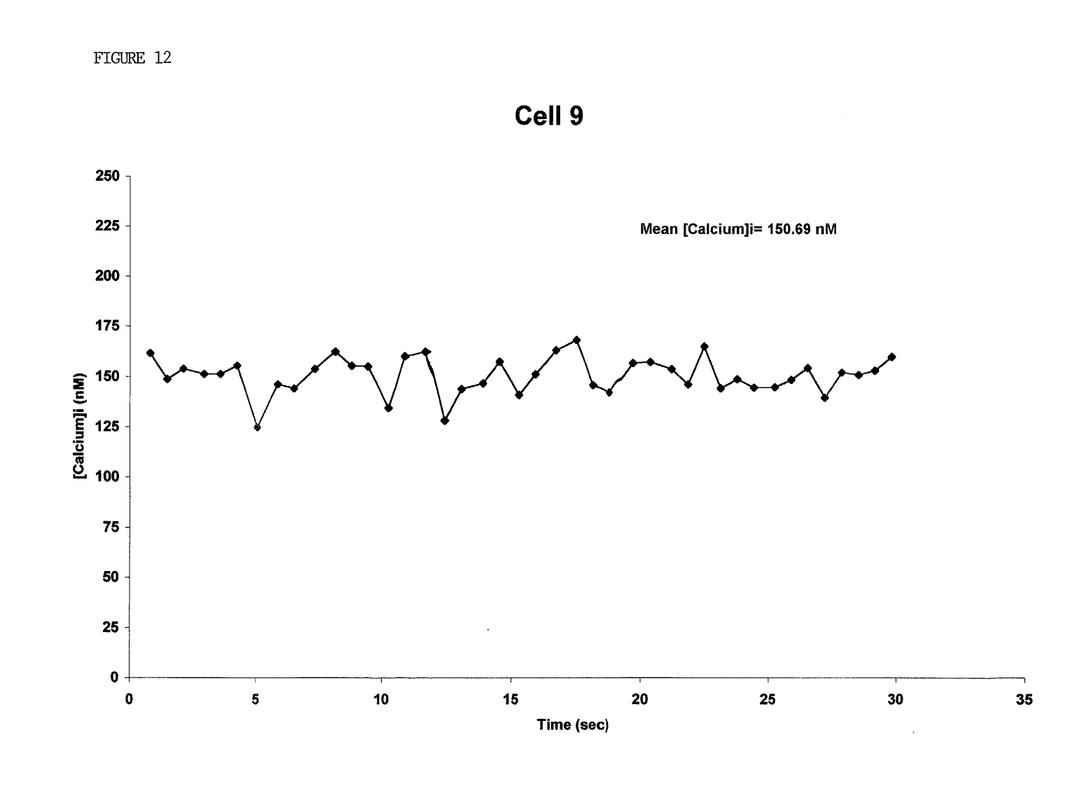

FIG. 12 (cell #9) shows the intracellular calcium concentration of an adult rat ventricular myocyte after 54 hours in culture. Calcium imaging data as measured using Fura-II (5 μM) (data not shown) shows that this cell is spontaneously contracting as evidenced by a shift in the calcium distribution.

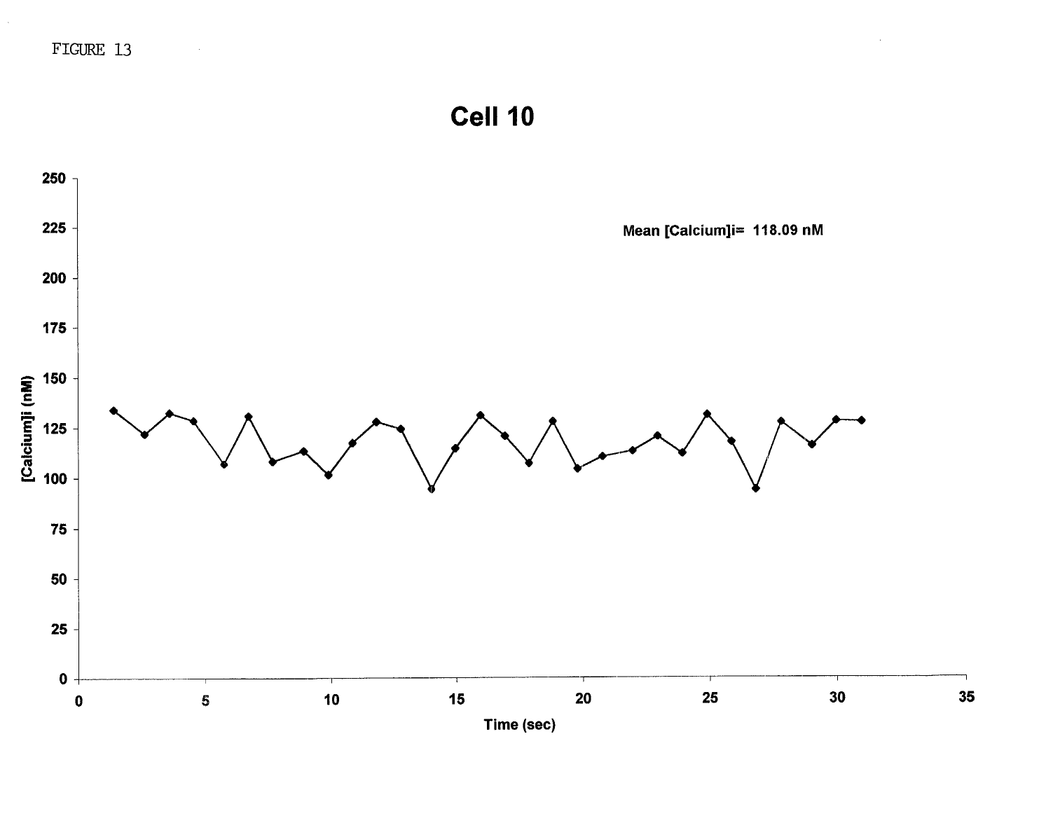

FIG. 13 (cell #10) is the same as cell #9. Calcium imaging data as measured using Fura-II (5 μM). This image shows that the quiescent cell has normal intracellular calcium distribution.

| [Calcium]_{I} | | | | |
|---|---|---|---|---|
| (nM) | Day 1 | Day 2 | Day 3 | Mean ± SEM |
| Normal quies- | 22.27 | 37.58 ± 9.47 | 15.50 ± 3.77 | 25.93 ± 5.71 |
| cent cells | | | | |
| Contracting | — | 95.21 | 134.39 ± 16.30 | 121.33 ± 16.10 |
| cells | | | | |

These data show that calcium tolerant ventricular myocytes from adult rats can be maintained for extended periods using our isolation procedures and maintenance (e.g., culture) media. The intracellular calcium measurements show that these cells have a steady and controlled calcium level throughout the time of maintenance and culture. The few cells that show higher intracellular calcium concentrations display spontaneous contraction. This indicates that unlike quiescent cells, contracting ones have their resting potential closer to the activation threshold for sodium or calcium channels (more depolarized). Thus, these cells are at a higher excitability level. In support of this, contracting cells show a higher basal level for intracellular calcium than quiescent ones. This demonstrates that the cells are intact and exhibit normal excitation-contraction coupling. Accordingly, the presently described cell isolation and maintenance/culture methods yield quiescent calcium-tolerant ventricular myocytes that can stably survive for at least 3 days.

### EXAMPLE 2

### Isolation/Maintenance of Human Cardiomyocytes

One gram of heart muscle is dissected from the left ventricular free wall and quickly chopped into chunks of approximately 1 mm³ using an array of razor blades. The chunks are incubated for 12 minutes, while shaking at 37° C. in 25 ml of a solution containing 1-2 μM calcium (LC) of the following composition in mM: NaCl 120; KCl 5.4; MgSO₄ 5; pyruvate 5; glucose 20; taurine 20; HEPES 10; and nitrilotriacetic acid 5, pH 6.96. The medium is changed several (˜3) times during the twelve minutes. The chunks are stirred by bubbling with 100% O₂. After removal of the LC medium by straining with 300 μm gauze, the chunks are incubated at 37° C. for 45 minutes in the above solution with nitrilotriacetic acid omitted and 4 U/ml of type XXIV protease and 30 μM calcium added, followed by two 45 minutes period with the protease omitted and 400 IU/ml collagenase added. The medium is shaken under an atmosphere of 100% O₂. At the end of the second and third 45 minute periods the solution containing the dispersed cells is filtered through a 300 μm gauze and centrifugated at 40 g for 1-2 min.

After isolation, the cells are washed in the same medium containing 30 μM calcium and resuspended in culturing media. Such culture media can comprise medium DMEM with the addition of 0.2 g BSA, 0.1 mM ascorbic acid, 50 mM taurine, 16 mM carnitine, 50 mM creatine, 0.1 μM insulin, 50 units/ml penicillin G sodium, 50 mg/ml streptomycin sulfate. Culture media can also comprise DMEM medium without calcium chloride anhydrous and D-calcium pantothenate. Sodium pantothenate substituted can be used in order to increase the calcium concentration in a cumulative manner to reduce calcium intolerance.

Omega 3 fatty acids have been shown by Kang & Leaf (Kang, J. X. and A. Leaf, Circulation (1996) 94(7): 1774-80) to protect against calcium overload and calcium paradox. Therefore, the culture media can also comprise omega fatty acids, such as, docosaheanoic acid, eicosapentaenoic acid, eicosatetraynoic acid, or polyunsaturated fatty acid.

Magnesium (Mg⁺) is also known to be protective against calcium overload and has been shown to be beneficial in failing human myocardium (Schwinger, R. H., et al., Am. Heart J. (1993) 126(4):1018-21; Schwinger, R. H., et al., J. Pharmacol. Exp. Ther. (1992) 263(3):1352-9). Therefore, the culture media can comprise varying concentrations of Mg²⁺, e.g., from 0.1 to 16 mM.

Isolated myocardiocytes resuspended in culture medium are infected with adenovirus at a multiplicity of infection (MOI) of 100 and incubated at 37° C. under an atmosphere of 95% O₂-5%CO₂ for 24 hrs or longer (as described below) for in vitro cell maintenance studies.

### EXAMPLE 3

### Viability of the Cells

In order to demonstrate that the cells are functionally intact and that calcium mobilization is not altered as a result of a change in the phenotype of the cells during cell maintenance, quantification of the intracellular calcium and shortening in freshly isolated myocytes is compared with cells maintained for 24, 48, 72 hours. The intracellular calcium determinations are particularly important as heart failure has been shown to significantly induce changes in key calcium regulatory proteins.

Cell shortening and calcium measurements: The isolated cells in suspension are loaded with the fluorescent indicator Fura 2 μM (Molecular Probes) at a concentration of 2 μM.

A drop of the cell suspension loaded with Fura 2 μM is placed in a chamber on the stage of an inverted microscope. The cells are then superfused with Krebs-Henseleit (K-H) solution containing 1.3 mM calcium equilibrated with 95% O₂-5%CO₂ and warmed to 32° C. The cells are electrically field stimulated with a biphasic pulse at 0.2 Hz, 50% above threshold through platinum electrodes placed along the side of the bath. The contraction amplitude and the rate of contraction and relaxation are monitored using a video edge detection system and data acquisition software (e.g., Ion Optix). The system uses a specially modified non-interlaced 60 Hz CCD camera that records the transmitted light image of the cell to be processed by the software to calculate the cell length. The software reads the standard 60 Hz image and calculates the length at each 240^{th} of a second.

Intracellular calcium is measured in the Fura 2 μM loaded cells under superfusion with K-H solution containing 500 μM probenecid with a dual-excitation spectrofluorometer (e.g., Ion Optix). The fluorescent images are recorded using a non-interlaced CCD camera, which produces 60 distinct 640×240 pixel images every second. The software pixel reading results in a 320×240 image thus in a 1:1 ratio. The camera and the chopperswitch light source are synchronized by the fluorescence system interface in order for the excitation light to occur at the start of each sequential camera image.

### EXAMPLE 4

### Biochemical Phenotyping—Protein and mRNA Comparison of Freshly Isolated Myocytes and Maintained Myocytes at Various Time Points to Snap Frozen Samples from the Same Hearts

At the time of harvesting, samples from each heart are snap frozen for later analysis.

Preparation of crude membranes: Left ventricular myocardial tissue is chilled in ice cold (4° C.) homogenization buffer with the following composition (in mmol/l): sucrose 300, PMSF 1, PIPES 20, pH 7.4. The homogenate (membrane) is spun at 8,000 rpm (Beckman JA 20) for 20 min. The supernatant is filtered through four layers of gauze. The suspension is then centrifuged at 37,000 rpm for 60 min at 4° C. (Beckman TI 70). The final pellet is resuspended in a buffer solution. The total protein concentration is measured according to the method of Lowry et al (Lowry, O. H., et al., J. Biol. Chem. (1951) 193: 265-275). To assess the similarity between the membrane preparations used, protein levels of the ryanodine receptors and calsequestrin in both failing and non-failing groups is measured.

Analytical gel electrophoresis and blotting techniques—Proteins: Tissue is homogenized in a HEPES buffered phosphate buffer (pH 7.4) with protease inhibitors and DTT added to preserve protein integrity. Cardiac samples are then solubilized in a 2% SDS solution with the protein content measured using the BCA method. Standard SDS-PAGE is performed in a cold room. Five to 15% SDS gels are employed as required for the particular protein of interest. Proteins are transferred to membranes using the semi-dry transfer protocol. Immuno blotting is performed using primary antibodies for SERCA2a, RYR-2, Phospholamban, Calsequestrin, DHP receptors and the Na⁺/Ca²⁺ exchanger. These markers have been selected based on known developmental changes and disease based expression patterns. Failing human hearts revert to “fetal” expression patterns for several key calcium regulatory proteins. The blots are then blocked with BSA and probed with a secondary antibody conjugated with alkaline phosphatase. The blots are analyzed by digital scanning followed by computer analyses (Kodak). In cases in which Western blotting reveals one major band, slot blot techniques are used as an alternative and potentially more quantitative technique. The specific protein levels thus determined are then expressed relative to the total protein content of the sample, or to the actin or GAPDH.

Analytical gel electrophoresis and blotting techniques—mRNA: Total cellular RNA is isolated from snap frozen LV ventricular tissues (snap frozen at the time of harvesting and the additional samples snap frozen upon arrival at the laboratory), as well as from cells that have been cultured for 24,48, 72 hours and longer using the standard guanidinium thiocyanate/phenol/chloroform extraction technique. The total RNA concentration is determined by spectrophotometry. The final RNA pellet is resuspended in RNAase free H₂O and stored at −80° C. until further analysis. RNA integrity is checked by agarose gel electrophoresis. Specific mRNA content in the samples is measured by agarose gel electrophoresis, blotting onto membrane, hybridization with ³²P labeled probes, autoradiography, digital scanning and analysis. 18S RNA and GAPDH mRNA serves as a reference. The specific probes used are directed to mRNA coding for the same proteins whose levels are measured by Western blotting described above in order to analyze the relation, if any, between gene expression and protein levels.

### EXAMPLE 5

### Construction and Characterization of Recombinant Adenoviral Vectors for in vitro Cell Maintenance Studies

Recombinant adenoviruses are constructed using HEK293 cells and E. coli cells, for example, by using the method described by Vogelstein and colleagues (He, T. C., et al., Proc. Natl. Acad. Sci. USA (1998) 95(5):2509-14).

E1-deleted and E1-E4 deleted Adenoviruses: For the generation of E1 deleted adenoviruses the pAdEasy-1 adenoviral plasmid (containing all Ad5 sequences except the E1 genes and part of the E3 genes) is used (provided by Dr. Vogelstein's laboratory) with the shuttle vector, pAdTRACK, containing green fluorescent protein, GFP, under the control of the CMV promoter and the promoter along with the cDNA of interest. These adenoviruses are propagated in HEK293 cells. The GFP insert identifies cardiomyocytes that have been infected and green fluorescence correlates with the physiological effects of the transgene. For the generation of E1-E4 deleted adenoviruses, the pAdEasy-2 adenoviral plasmid (similar to pAdEasy-1 except that it contains an additional deletion encompassing part of the E4 gene) is used. The E1-E4 deleted adenoviruses is propagated in an E4 expressing cell line (911 cell line). In general, E1 deleted adenoviruses are much easier to generate and to grow when compared to E1-E4 deleted adenoviruses.

Tissue Specific Promoters: Since non-specific tissue expression is a limitation for in vivo cardiac gene transfer, a tissue-specific promoter for cardiac gene transfer, namely the 250 bp fragment of the myosin light chain-2v (MLC-2v) gene which is known to be expressed in a tissue-specific manner in ventricular myocardium, is used. An adenovirus is constructed containing 250 bp fragment of the MLC-2v promoter as well as a construct containing 4x this MLC-2v promoter fragment controlling the expression of the reporter gene luciferase (Ad.1xMLC-2v.Luc and Ad.4xMLC-2v.Luc). As a positive control, an adenovirus containing luciferase controlled by a CMV promoter is constructed (Ad.CMV.Luc). A promoter-less adenovirus with luciferase is used as a negative control (Ad.Ø.Luc). When injected into the left ventricular wall of rats as well as into the middle lobe of the liver, Ad.1xMLC.Luc shows significantly higher activity of luciferase (2,400-fold increase; p<0.00001) in vivo as compared to Ad.Ø.Luc (n=6) and has 24.4% activity compared to Ad.CMV.Luc infected ventricles. Ad.4xMLC.Luc has slightly less expression than Ad.1xMLC.Luc in the left ventricle in vivo (16.1% vs 24.4%), but showed significantly lower expression in the injected liver tissue (60,000 vs 1,500,000 RLU (relative light units); p<0.0001). The heart/liver ratio is significantly higher in Ad.4xMLC.Luc than Ad.1xMLC.Luc (46.03 vs. 2.8). Both E1 deleted and E1-E4 deleted adenoviruses with 4xMLC are used for transfection of maintained human myocytes.

Adenoviral infection: The efficiency of adenoviral gene transfer is evaluated in myocytes using Ad.CMV.βgal.GFP which has a dual cassette for β-gal and GFP under the control of separate CMV promoters. The reporter adenovirus Ad.CMV.βgal.GFP is added at a Multiplicity of Infection (MOI) of 1, 10, 50 and 100 pfu/cell. Cells are incubated in media conditions used to prolong the survival of the cardiomyocytes at 37° C. Twenty-four, 48, and 72 hours after in vitro exposure to Adβgal, the myocytes are fixed in 0.05% glutaraldehyde for 5 min at room temperature. The cells are then stained overnight at 37° C. in PBS containing 1.0 mg/ml 5-bromo-4-chloro-3-indolyl β-D-galactopyranoside (X-gal), 15 mM potassium ferricyanide, 15 mM potassium ferrocyanide, and 1 mM MgCl₂. With this reaction, cells that stain blue express the β-galactosidase. The efficiency of the infection is assessed by counting the number of blue-staining cells per high power field. For accurate counts, approximately 500 cells are counted in each dish at a minimum of ten dishes per heart.

Results show that the efficiency of gene transfer is significant starting at a virus concentration of 10 plaque forming units/cell with 100% of the cells being infected at 100 pfu/cell. In a similar manner, human ventricular myocardial cells are infected with three concentrations of the adenoviral vectors carrying the transgene: 10, 50, and 100 pfu/cell in the various media conditions. Results show that, infection with either Ad.RSV.βgal or Ad.RSV.SERCA2a does not change the morphology of human cells.

Affects on (1) cell morphology and structure, (2) contractility, (3) survival, and (4) changes in expression pattern of key proteins are quantified.

### EXAMPLE 6

### Effects of Adenoviral Gene Transfer on Isolated Human Ventricular Cardiomyocytes in vitro

Human ventricular adult cardiomyocytes are infected with adenoviral vector comprising a green fluorescent protein (Ad.GFP). Infected cardiomyocytes show green fluorescence under fluorescence light. Human cardiomyocytes isolated from the ventricle of a human patient with dilated cardiomyopathy and infected with Ad.GFP (100 pfu/cell) survived for six (6) days without changes in the rod shaped form and myofibrillar striations while expressing GFP (data not shown).

In order to obtain the target protein expression following the viral infection, a preservation period long enough is essential. Using DMEM culture medium without serum, but with creatine, camitine, taurine, insulin and BSA, myocyte numbers are well preserved at 24 h, declining to about 50% at 48h.

Rod-shaped morphology is preserved and shows infection and expression of the reporter gene as well as the sarcoplasmic reticulum (SR) Ca⁺⁺ ATPase (SERCA2a), with preservation of the contraction characteristics of the myocytes. Application of adenovirus containing the reporter gene green fluorescent protein (GFP) demonstrates efficient infection of the myocytes, with up to 95% of the viable cells expressing protein at 24 hour.

### EXAMPLE 7

### Contractile Response and Calcium Transient of Isolated Human Myocytes After Infection with Ad.GFP or Ad.SERCA2a

Contractile function is unchanged at 24 h in terms of contraction amplitude, time-to-peak contraction (TTP), time-to-50% relaxation (R50), frequency-dependent changes in amplitude and responses to increasing extracellular Ca⁺⁺ concentrations. By 48 hrs changes are occurring, especially to relaxation times, although myocytes are still responsive to multiple challenges with high extracellular Ca⁺⁺ concentration.

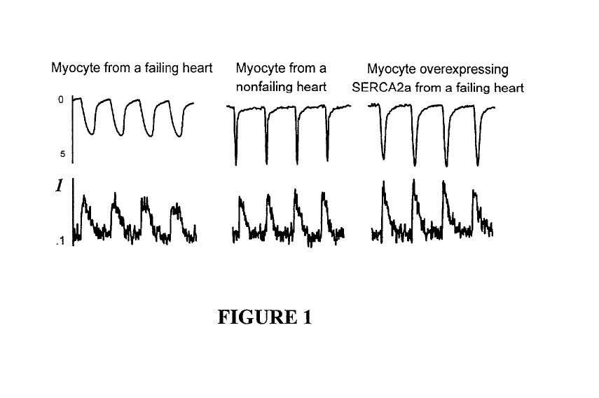

Results with adenovirus encoding for SERCA2a show that it is possible to express sufficient protein in 24 h to affect function. FIG. 1 shows a comparison of cell shortening and calcium transients from a failing and a non-failing isolated human myocyte overexpressing GFP, and a failing myocyte overexpressing SERCA2a. As seen in FIG. 1, recordings from cardiomyocytes isolated from donor nonfailing heart and from failing heart infected with either Ad.GFP or Ad.SERCA2a are compared as stimulated at 1 Hz at 37° C. Failing cells show a characteristic decrease in contraction and prolonged relaxation along with a prolonged Ca²⁺ transient. Overexpression of SERCA2a in failing cardiomyocyte normalized these parameters (del Monte, F., et al., Circulation (1999) 100(23):2308-11).

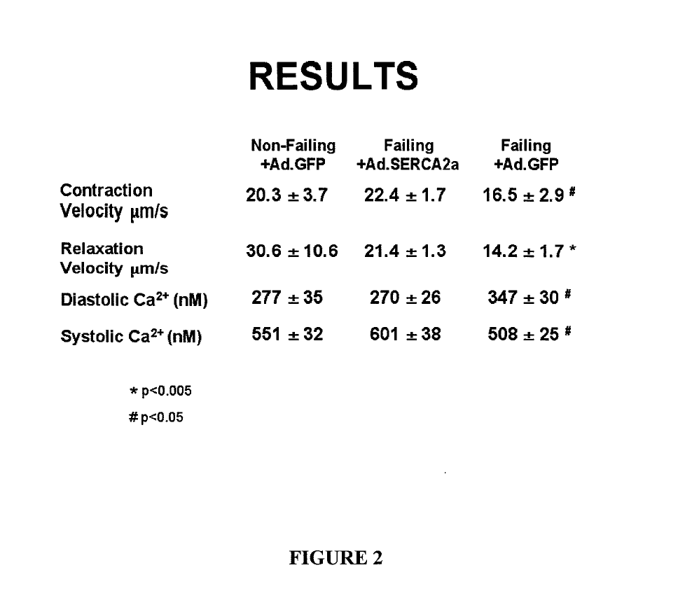

FIG. 2 shows contraction velocity, relaxation and systolic and diastolic Ca²⁺ concentrations in human cardiomyocytes from a donor nonfailing heart and from a failing heart infected with either Ad.GFP or Ad.SERCA2a, stimulated at 1 Hz at 37° C. (del Monte, F., et al., Circulation (1999) 100(23):2308-11).

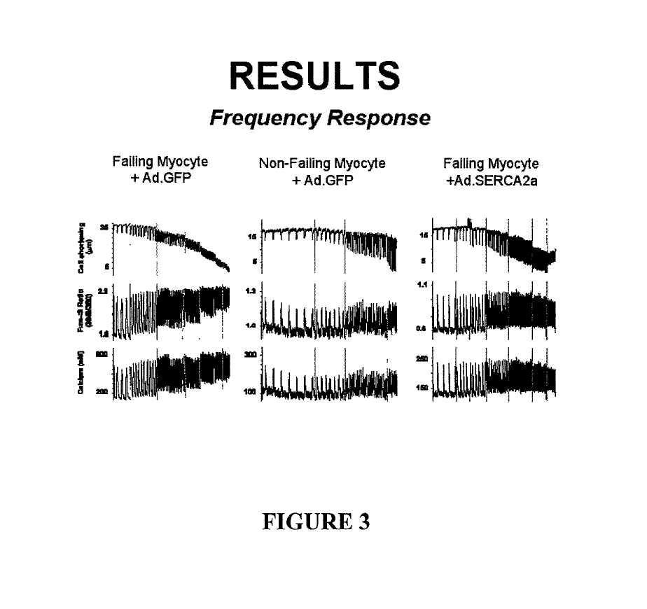

FIG. 3 shows recordings from the same cardiomyocytes as in FIG. 2 stimulated at increasing frequencies. Failing cardiomyocyte demonstrated a decrease in contraction amplitude and an increase in diastolic tone and Ca²⁺. Overexpression of SERCA2a restored frequency-dependent increase in contraction amplitude and mitigated an increase in diastolic Ca²⁺ and decreased length (del Monte, F., et al., Circulation (1999) 100(23):2308-11).

### Equivalents

Those skilled in the art will recognize, or be able to ascertain using no more than routine experimentation, many equivalents to specific embodiments of the invention described specifically herein. Such equivalents are intended to be encompassed in the scope of the following claims.

What is claimed is:

1. A method of isolating cells comprising,
(a) obtaining a tissue sample from a subject,
(b) successively exposing the tissue to a first solution with decreasing amounts of CaCl₂ comprising NaCl, HEPES, MgCl₂, KCl, and sugar at a pH of approximately 7.4,
(c) disassociating the tissue with an enzyme solution,
(d) repeatedly resuspending the disassociated tissue into a second solution with increasing amounts of CaCl₂ comprising Earle's modified salt, L-glutamine, sodium bicarbonate, sodium pentothenate, creatine, taurine, ascorbic acid, HEPES, fetal bovine serum, an antibiotic, and a fatty acid, at a pH of approximately 7.4 to obtain isolated cells.

2. The method of claim 1, further comprising the step of re-suspending the isolated cells approximately every 24 hours in a solution comprising Earle's modified salt, L-glutamine, sodium bicarbonate, sodium pentothenate, creatine, taurine, ascorbic acid, HEPES, fetal bovine serum, an antibiotic, a fatty acid acid, and CaCl₂ at a pH of approximately 7.4.

3. The method of claim 1, further comprising the step of incubating the isolated cells in a mixture of carbon dioxide and air.

4. The method of claim 3, wherein the isolated cells are incubated at approximately 37° C.

5. The method of claim 1 wherein, the first solution is exposed to the tissue at approximately 37° C. and at approximately 4 ml/min for 3 minutes.

6. The method of claim 1 wherein the concentration of CaCl₂ in the first solution decreases.

7. The method of claim 1 wherein the first solution comprises approximately 140 mM NaCl, approximately 10 mM HEPES, approximately 1 mM MgCl₂, approximately 5.4 mM KCl, and approximately 10 mM D-glucose.

8. The method of claim 1 wherein the enzyme solution comprises a digestive enzyme.

9. The method of claim 8, wherein the digestive enzyme is a protease or a collagenase.

10. The method of claim 1 wherein the concentration of CaCl₂ in the second solution increases.

11. The method of claim 1 wherein the enzyme solution comprises approximately 140 mM NaCl, approximately 10 mM HEPES, approximately 1 mM MgCl₂, approximately 5.4 mM KCl, and approximately 10 mM D-glucose.

12. The method of claim 1 wherein the second solution comprises Earle's modified salt, L-glutamine, sodium bicarbonate at approximately 1250 mg/l, sodium pentothenate, creatine at approximately 328 mg/500 ml, taurine at approximately 312 mg/500 ml, Ascorbic acid at approximately 8.8 mg, HEPES at approximately 2.383 g/500 ml, fetal bovine serum at approximately 10% v/v, an antibiotic at approximately 5% v/v, a fatty acid at approximately 1 μM at a pH of approximately 7.4.

13. A method of isolating cells comprising,
(a) obtaining a tissue sample from a subject,
(b) successively exposing at approximately 37° C. the tissue to a first solution with decreasing amounts of CaCl₂ comprising approximately 140 mM NaCl, approximately 10 mM HEPES, approximately 1 mM MgCl₂, approximately 5.4 mM KCl, and approximately 10 mM sugar at a pH of approximately 7.4,
(c) disassociating the tissue with an enzyme solution for approximately 8 minutes comprising approximately 140 mM NaCl, approximately 10 mM HEPES, approximately 1 mM MgCl₂, approximately 5.4 mM KCl, and approximately 10 mM sugar, to form disassociated cells,
(d) repeatedly resuspending the disassociated cells into a second solution with increasing amounts of CaCl₂ comprising Earle's modified salt, L-glutamine, sodium bicarbonate at approximately 1250 mg/l, sodium pentothenate, creatine at approximately 328 mg/500 ml, taurine at approximately 312 mg/500 ml, ascorbic acid at approximately 8.8 mg, HEPES at approximately 2.383 g/500 ml, fetal bovine serum at approximately 10% v/v, an antibiotic at approximately 5% v/v, and a fatty acid at approximately 1 μM at a pH of approximately 7.4 to form a solution of isolated cells,
(e) incubating the isolated cells in a mixture of carbon dioxide and air at approximately 37° C., and
(f) re-suspending the isolated cells approximately every 24 hours in a solution comprising Earle's modified salt, L-glutamine, sodium bicarbonate, sodium pentothenate, creatine, taurine, ascorbic acid, HEPES, fetal bovine serum, an antibiotic, a fatty acid, and CaCl₂ at a pH of approximately 7.4 to obtain isolated cells.

14. A method of cultivating isolated cells comprising, resuspending the isolated cells approximately every 24 hours in a solution comprising Earle's modified salt, L-glutamine, sodium bicarbonate, sodium pentothenate, creatine, taurine, ascorbic acid, HEPES, fetal bovine serum, an antibiotic, a fatty acid, and CaCl₂ at a pH of approximately 7.4.

15. The method of claim 14 wherein the solution comprises sodium bicarbonate at approximately 1250 mg/l, creatine at approximately 328 mg/500 ml, taurine at approximately 312 mg/500 ml, ascorbic acid at approximately 8.8 mg/500 ml, HEPES at approximately 2.383 g/500 ml, fetal bovine serum at approximately 10% v/v, an antibiotic at approximately 5% v/v, and a fatty acid at approximately 1 μM, and approximately 1 mM CaCl₂.

16. A cell culture media for cells comprising Earle's modified salt, L-glutamine, sodium bicarbonate, sodium pentothenate, creatine, taurine, ascorbic acid, HEPES, fetal bovine serum, an antibiotic, a fatty acid, and CaCl₂ at a pH of approximately 7.4.

17. The cell culture media of claim 16 wherein the media comprises sodium bicarbonate at approximately 1250 mg/l, creatine at approximately 328 mg/500 ml, taurine at approximately 312 mg/500 ml, ascorbic acid at approximately 8.8 mg/500 ml, HEPES at approximately 2.383 g/500 ml, fetal bovine serum at approximately 10% v/v, an antibiotic at approximately 5% v/v, a fatty acid at approximately 1 μM, and approximately 1 mM CaCl₂.

18. A method of isolating cells comprising,
(a) obtaining a tissue sample comprising cells from a subject;
(b) chopping the tissue;
(c) incubating the tissue in a first solution comprising calcium, salts, magnesium sulfate, pyruvate, glucose, taurine, HEPES, and nitrilotriacetic acid;
(d) incubating the tissue in a second solution comprising calcium, salts, magnesium sulfate, pyruvate, glucose, taurine, HEPES, and a digestive enzyme;
(e) incubating the tissue in a third solution comprising calcium, salts, magnesium sulfate, pyruvate, glucose, taurine, HEPES, and a digestive enzyme; and
(f) centrifuging the tissue to obtain isolated cells.

19. The method of claim 18, further comprising the step of resuspending the isolated cells in a culture media comprising medium M199, BSA, ascorbic acid, taurine, carnitine, creatinine, insulin, and an antibiotic.

20. The method of claim 19, wherein the culture media further comprises a fatty acid or magnesium.

21. The method of claim 18, wherein the first solution comprises approximately 1-2 μM CaCl₂, approximately 120 mM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and approximately 5 mM nitrilotriacetic acid, at a pH of approximately 6.96.

22. The method of claim 18, wherein the second solution comprises approximately 1-2 μM CaCl₂, approximately 30 μM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and 4 U/ml of a digestive enzyme.

23. The method of claim 18, wherein the third solution comprises approximately 1-2 μM CaCl₂, approximately 30 μM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and 4 U/ml of a digestive enzyme.

24. A method of isolating cells comprising,
(a) obtaining a tissue sample comprising cells from a subject;
(b) chopping the tissue;
(c) incubating the tissue in a first solution comprising approximately 1-2 μM CaCl₂, approximately 120 mM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and approximately 5 mM nitrilotriacetic acid, at a pH of approximately 6.96;
(d) shaking the tissue at approximately 37° C. for approximately 12 minutes;
(e) bubbling approximately 100% O₂ through the solution;
(f) incubating the tissue in a second solution comprising approximately 1-2 μM CaCl₂, approximately 30 μM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and 4 U/ml of a digestive enzyme;
(g) incubating the solution in a third solution comprising third solution comprises approximately 1-2 μM CaCl₂, approximately 30 μM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and 4 U/ml of a digestive enzyme; and
(h) centrifuging the tissue to obtain isolated cells.

25. A method of isolating and cultivating human myocardial cells comprising,
(a) obtaining a tissue sample comprising myocardial cells from a human subject;
(b) chopping the tissue;
(c) incubating the tissue in a first solution comprising approximately 1-2 μM calcium, approximately 120 mM NaCl, approximately 5.4 mM KCl, approximately 5 mM MgSO₄, approximately 5 mM pyruvate, approximately 20 mM glucose 20, approximately 20 mM taurine, approximately 10 mM HEPES, and approximately 5 mM nitrilotriacetic acid, at a pH of approximately 6.96;
(d) shaking the tissue at approximately 37° C. for approximately 12 minutes;
(e) bubbling approximately 100% O₂ through the solution;
(f) incubating the tissue in a second solution comprising approximately 1-2 μM, approximately 30 μM NaCl, approximately 5.4 mM KCl, approximately 5 mM MgSO₄, approximately 5 mM pyruvate, approximately 20 mM glucose 20, approximately 20 mM taurine, approximately 10 mM HEPES, and 4 U/ml of a digestive enzyme;
(g) incubating the solution in a third solution comprising third solution comprises approximately 1-2 μM, approximately 30 μM NaCl, approximately 5.4 mM KCl 5.4, approximately 5 mM MgSO₄, approximately 5 mM pyruvate, approximately 20 mM glucose 20, approximately 20 mM taurine, approximately 10 mM HEPES, and 400U/ml of a digestive enzyme;
(h) centrifuging the tissue to obtain isolated cells;
(i) repeatedly resuspending the disassociated cells into a second solution which comprises increasing amounts of CaCl₂, Earle's modified salt, L-glutamine, sodium bicarbonate at approximately 1250 mg/l, sodium pentothenate, creatine at approximately 328 mg/500 ml, taurine at approximately 312 mg/500 ml, ascorbic acid at approximately 8.8 mg, HEPES at approximately 2.383 g/500 ml, fetal bovine serum at approximately 10% v/v, an antibiotic at approximately 5% v/v, and a fatty acid at approximately 1 μM at a pH of approximately 7.4 to form a solution of isolated cells; and
(j) incubating the isolated cells in a mixture of carbon dioxide and air at approximately 37° C.

26. A method of isolating and cultivating rodent myocardial cells comprising,
(a) removing the heart of a rodent;
(b) perfusing the heart with low calcium Tyrode's solution for approximately 3 minutes;
(c) perfusing the heart with an enzymatic solution for approximately 8 minutes;
(d) perfusing the heart with a low calcium solution for approximately 3 minutes;
(e) removing the ventricles;
(f) mincing the ventricles to isolate myocardial cells;
(g) mixing the cells in a low calcium solution;
(h) resuspending the cells in a solution comprising increasing concentrations of calcium; and
(i) resuspending the cells in culture media solution..
